(19) United States
(12) Patent Application Publication
(10) Pub. No.: US 2023/0416472 A1
(43) Pub. Date: Dec. 28, 2023

(54) PHOTO-CROSS-LINKING COPOLYMERIZED HYALURONIC ACID SPONGE AND PREPARATION METHOD THEREFOR

(71) Applicant: SNVIA CO., LTD., Busan (KR)

(72) Inventors: Sam Dae PARK, Busan (KR); Su Young PARK, Busan (KR); Young Jun HWANG, Busan (KR); Se Min JEONG, Busan (KR)

(73) Assignee: SNVIA CO., LTD., Busan (KR)

(21) Appl. No.: 18/037,997

(22) PCT Filed: Nov. 15, 2021
(86) PCT No.: PCT/KR2021/016590

§ 371 Date: May 19, 2023

(30) Foreign Application Priority Data
Nov. 20, 2020 (KR) 10-2020-0156125

(51) Int. Cl. C08J 3/28; C08J 3/24; C08L 5/08; C08J 9/28; A61L 15/28; A61L 15/42; A61L 15/64

(52) U.S. Cl. CPC C08J 3/28; C08J 3/24; C08L 5/08; C08J 9/28; A61L 15/28; A61L 15/425; A61L 15/64; C08J 2305/08

(57) ABSTRACT

There are provided a photo-cross-linking copolymerized hyaluronic acid sponge and a preparation method therefor, the sponge not being sticky and not dripping, having flexibility to be usable in various parts of the body, having excellent water binding force to help tissue regeneration, having very excellent biocompatibility so as to be absorbable into the skin when attached to the body for a long time, so that the trouble of having to be removed can be decreased, and comprising, through a photo-cross-linking reaction, polymers having various lengths so as to be applicable in various forms of medical antihemorrhagic agents, wound-coating agents, adhesive barriers.

### CROSS-REFERENCE TO PRIOR APPLICATIONS

This application is a National Stage Patent Application of PCT International Patent Application No. PCT/KR2021/016590 (filed on Nov. 15, 2021) under 35 U.S.C. § 371, which claims priority to Korean Patent Application No. 10-2020-0156125 (filed on Nov. 20, 2020), which are all hereby incorporated by reference in their entirety.

### BACKGROUND

The present invention relates to a photo-cross-linking copolymerized hyaluronic acid sponge and a preparation method therefor, and, more specifically, to a photo-cross-linking copolymerized hyaluronic acid sponge and a preparation method therefor, the sponge not being sticky and not dripping, having flexibility to be usable in various parts of the body, having excellent water binding force to help tissue regeneration, having very excellent biocompatibility so as to be absorbable into the skin when attached to the body for a long time, so that the trouble of having to be removed can be decreased, and comprising, through a photo-cross-linking reaction, polymers having various lengths so as to be applicable in various forms of medical antihemorrhagic agents, wound-coating agents, adhesive barriers, and the like.

Recently, many studies have been conducted on absorbent materials in the form of sponges prepared by cross-linking and freeze-drying of biodegradable polymers in the field of surgery and wound healing. These materials have high biocompatibility and are cross-linked and so they can retain moisture for a long time and are not toxic when absorbed. Therefore, they are useful for wound healing both inside and outside the body.

In particular, hyaluronic acid (HA), which is a component of various tissues in the human body, can help in wound healing, and the introduction of a cross-linking group can help in tissue regeneration as well as wound suture.

However, most of the existing cross-linked hyaluronic acid can only be used as a gel formulation, and the application thereof is limited due to gel's stickiness and dripping. Even when it is prepared as a patch and the like, the flexibility is decreased and the control of the properties is difficult, and so it is applicable to only limited fields.

To overcome the problems described above, the inventors of the present invention have developed a sponge-type photo-cross-linked hyaluronic acid that does not cause discomfort such as stickiness and dripping. However, previously developed products had little softness and so they were limited in the application to curved parts of the body.

Therefore, the inventors of the present invention recognized the urgent need for developing a sponge-type photo-cross-linking copolymerized hyaluronic acid with flexibility for application to various curved parts of the body and completed the present invention.

### SUMMARY

The purpose of the present invention is to provide a photo-cross-linking copolymerized hyaluronic acid sponge and a preparation method therefor, the sponge not being sticky and not dripping, having flexibility to be usable in various parts of the body, having excellent water binding force to help tissue regeneration, having very excellent biocompatibility so as to be absorbable into the skin when attached to the body for a long time, so that the trouble of having to be removed can be decreased.

Another purpose of the present invention is to provide a photo-cross-linking copolymerized hyaluronic acid sponge and a preparation method therefor, the sponge comprising, through a photo-cross-linking reaction, polymers having various lengths so as to be applicable in various forms of medical antihemorrhagic agents, wound-coating agents, adhesive barriers, and the like.

The technical problems of the present invention are not limited to the technical problems mentioned above, and other technical problems that are not mentioned will be clearly understood by one of ordinary skill in the art from the description below.

To achieve the purpose described above, the present invention provides a photo-cross-linked hyaluronic acid sponge, a preparation method therefor, and a use thereof.

Hereinafter, the present specification will be described in more detail.

The present invention provides a photo-cross-linking copolymerized hyaluronic acid sponge comprising a compound represented by [Formula 1] below.

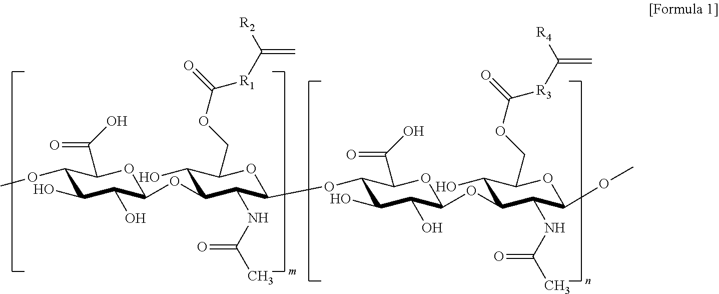

wherein, in [Formula 1] above,
the R1 and R3 are each independently a C₀-C₇ alkyl group,
the R2 and R4 are each independently a C₀-C₁ alkyl group, and
the m and n are each independently a positive integer of 1 to 10,000.

In the present invention, the compound represented by [Formula 1] is characterized in that it is a compound represented by [Formula 2] below:

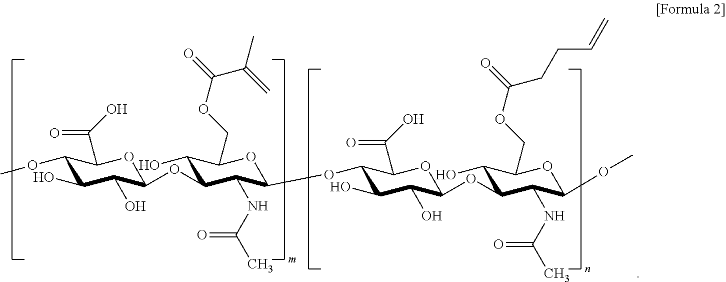

In addition, the present invention provides a preparation method for a photo-cross-linked hyaluronic acid sponge comprising steps described below:

(S1) preparing a compound represented by [Formula 1] below; and

(S2) preparing a photo-cross-linked hyaluronic acid sponge by adding a photoinitiator to the compound represented by the [Formula 1].

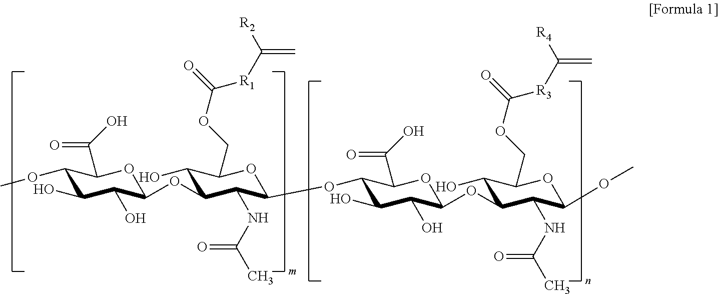

wherein, in [Formula 1] above,
the R1 and R3 are each independently a C₀-C₇ alkyl group,
the R2 and R4 are each independently a C₀-C₁ alkyl group, and
the m and n are each independently a positive integer of 1 to 10,000.

In the present invention, the step (S1) is characterized in that it consists of steps described below:

(S1A) preparing a first solution by dissolving hyaluronic acid (HA) in water (H₂O);

(S1B) preparing a second solution by adding a compound having a photo-cross-linking functional group and a base to the first solution; and

(S1C) preparing a compound represented by [Formula 1] by adding an alcohol to the second solution to form a precipitate.

In the present invention, the compound having a photo-cross-linking functional group is at least one selected from the group consisting of methacrylic anhydride, acrylic anhydride, 3-butenoic anhydride, 4-pentenoic anhydride, 5-hexenoic anhydride, 6-heptenoic anhydride, 7-octenoic anhydride, 8-nonenoic anhydride, and 9-decenoic anhydride.

In the present invention, the compound represented by [Formula 1] above is characterized in that it is a compound represented by [Formula 2] below:

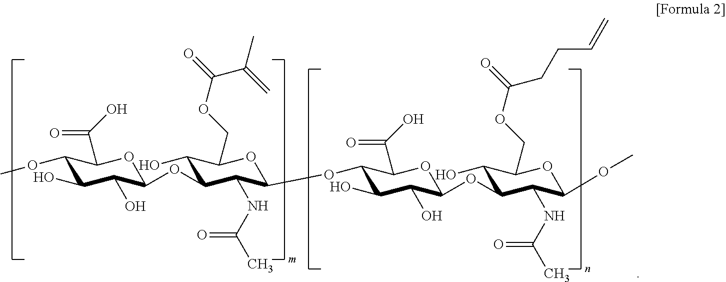

In the present invention, the step (S2) is characterized in that it consists of steps described below:

(S2A) preparing an aqueous solution by adding a photoinitiator and water (H₂O) to the compound represented by [Formula 1] above;

(S2B) irradiating ultraviolet (UV) ray to the aqueous solution to perform a photo-cross-linking reaction; and

(S2C) after the photo-cross-linking reaction, freeze-drying at −40 to 40° C. to prepare a photo-cross-linked hyaluronic acid sponge.

In the present invention, the photoinitiator is characterized in that it is at least one selected from the group consisting of 2-hydroxy-1-[4-(2-hydroxyethoxy)phenyl] 2-methyl-1-propanone, 3-(4-benzoylphenoxy)-2-hydroxy-N,N,N-trimethyl-1-propanaminium-chloride, methyl diethanolamine, sodium phenyl-2,4,6-trimethylbenzoylphosphinate, lithium phenyl-2,4,6-trimethylbenzoylphosphinate, benzyldimethyl ketal, and 1-hydroxycyclohexyl phenyl ketone.

All matters mentioned in a photo-cross-linked hyaluronic acid sponge and a preparation method therefor of the present invention are equally applied unless they contradict with each other.

The photo-cross-linking copolymerized hyaluronic acid sponge and the preparation method therefor of the present invention, the sponge not being sticky and not dripping, having flexibility to be usable in various parts of the body, having excellent water binding force to help tissue regeneration, and having very excellent biocompatibility so as to be absorbable into the skin when attached to the body for a long time, can decrease the trouble of having to remove.

In addition, the photo-cross-linking copolymerized hyaluronic acid sponge and the preparation method therefor of the present invention, the sponge comprising, through a photo-cross-linking reaction, polymers having various lengths, can be usefully applied in various forms of medical antihemorrhagic agents, wound-coating agents, adhesive barriers, and the like.

The effects of the present invention are not limited to the effects mentioned above, and other effects that are not mentioned will be clearly understood by one of ordinary skill in the art from the description of the claims.

### BRIEF DESCRIPTION OF THE DRAWINGS

FIG. 1 is a block diagram schematically showing the preparation method for a photo-cross-linking copolymerized hyaluronic acid sponge of the present invention.

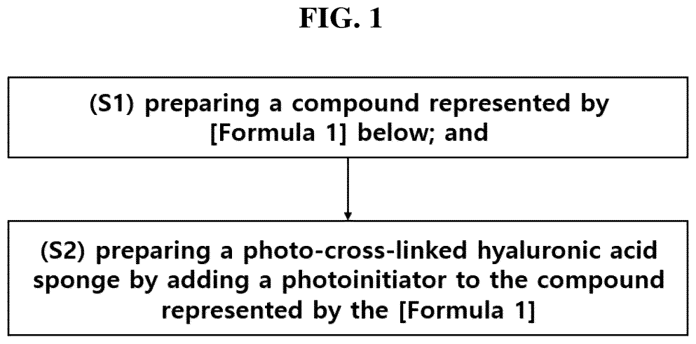

FIG. 2 is a ¹H NMR spectrum of the compound represented by [Formula 2] prepared in Example 1 according to the present invention.

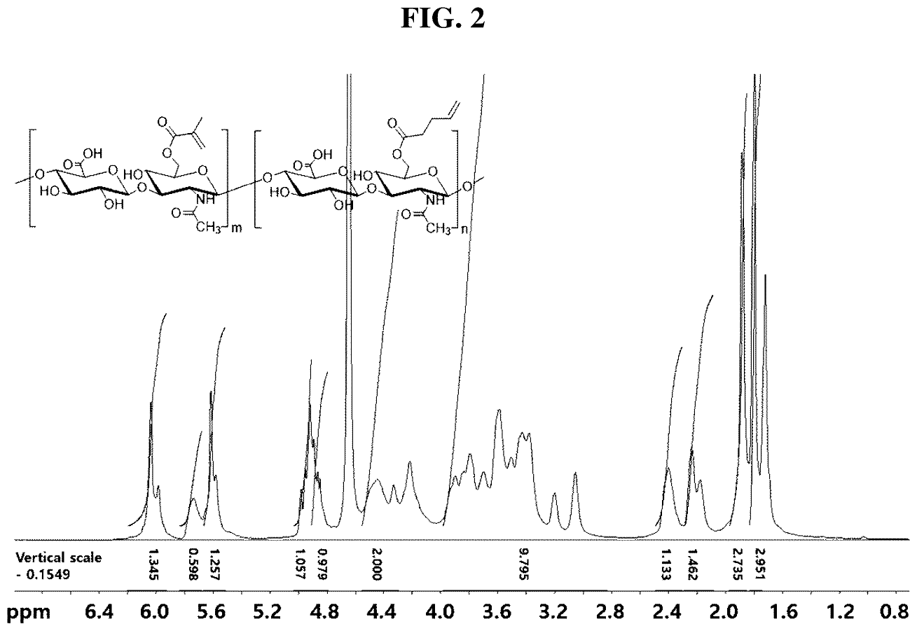

FIG. 3 is an image confirming the water binding force and flexibility of the photo-cross-linking copolymerized hyaluronic acid sponge prepared in Example 1 according to the present invention and the comparative photo-cross-linking copolymerized hyaluronic acid sponge prepared in Comparative Example 1.

### DETAILED DESCRIPTION

The terms used in this specification have been selected from general terms that are widely used at present as far as possible in consideration of the functions in the present invention, but these may differ depending on the intention of one of ordinary skill in the art, the precedent, or the emergence of new technologies. In addition, in some specific cases, terms were arbitrarily selected by the applicant, and in these cases, the meaning will be described in detail in the detailed description of the present invention. Therefore, the term used in the present invention should be defined based on the meaning of the term and the overall content of the present invention, not simply the name of the term.

Unless defined otherwise, all terms used herein, including technical or scientific terms, have the same meaning as commonly understood by one of ordinary skill in the art to which the present invention belongs. Terms such as those defined in commonly used dictionaries should be interpreted as having a meaning consistent with the meaning in the context of the related art, and unless explicitly defined in the present application, the terms should not be interpreted in an ideal or excessively formal meaning.

Numerical ranges are inclusive of the values defined therein. Every maximum numerical limitation given throughout the present specification includes every lower numerical limitation, as if a lower numerical limitation were explicitly described. Every minimum numerical limitation given throughout the present specification includes every higher numerical limitation, as if a higher numerical limitation were explicitly described. Every numerical limitation given throughout the present specification will include every better numerical range within a broader numerical range, as if a narrower numerical limitation were explicitly described.

Hereinafter, Examples of the present invention will be described in detail, but it is obvious that the present invention is not limited by the Examples described below.

### Photo-Cross-Linking Copolymerized Hyaluronic Acid Sponge

The present invention relates to a photo-cross-linking copolymerized hyaluronic acid sponge, and more specifically, a photo-cross-linking copolymerized hyaluronic acid sponge comprising a compound represented by [Formula 1] below:

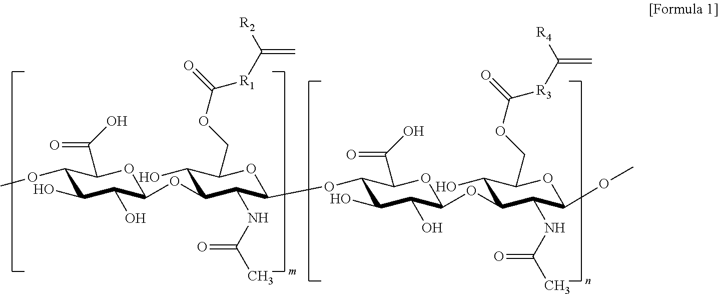

wherein, in [Formula 1] above,
the R1 and R3 are each independently a C₀-C₇ alkyl group,
the R2 and R4 are each independently a C₀-C₁ alkyl group, and
the m and n are each independently a positive integer of 1 to 10,000.

The compound represented by [Formula 1] above may have a structure in which hyaluronic acid is a precursor and a photo-cross-linkable functional group is added to the hyaluronic acid, and more specifically, the compound represented by [Formula 1] above may be a compound represented by [Formula 2] below:

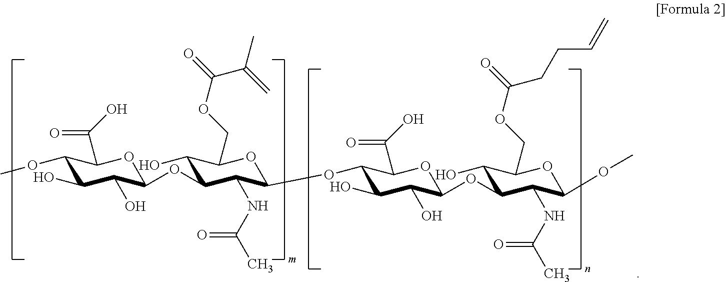

### Preparation Method for Photo-Cross-Linking Copolymerized Hyaluronic Acid Sponge

The present invention provides a preparation method for a photo-cross-linking copolymerized hyaluronic acid sponge comprising steps described below:

(S1) preparing a compound represented by [Formula 1] below; and

(S2) preparing a photo-cross-linking copolymerized hyaluronic acid sponge by adding a photoinitiator to the compound represented by the [Formula 1].

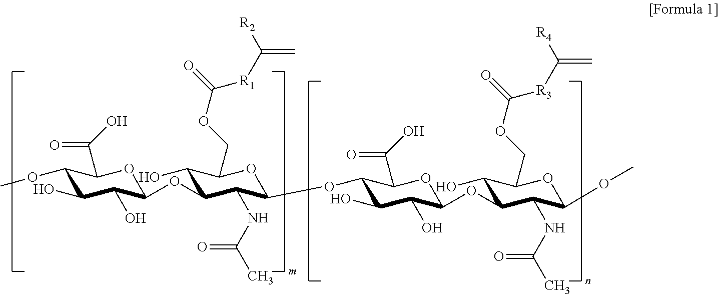

wherein, in [Formula 1] above,
the R1 and R3 are each independently a C₀-C₇ alkyl group,
the R2 and R4 are each independently a C₀-C₁ alkyl group, and
the m and n are each independently a positive integer of 1 to 10,000. The step (S1), which is a step for preparing a compound represented by [Formula 1], may consisting of steps described below:

(S1A) preparing a first solution by dissolving hyaluronic acid (HA) in water (H₂O);

(S1B) preparing a second solution by adding a compound having a photo-cross-linking functional group and a base to the first solution; and

(S1C) preparing a compound represented by [Formula 1] by adding an alcohol to the second solution to form a precipitate.

The compound represented by [Formula 1] above may have a structure in which hyaluronic acid is a precursor and a photo-cross-linkable functional group is added to the hyaluronic acid, and more specifically, the compound represented by [Formula 1] above may be a compound represented by [Formula 2] below:

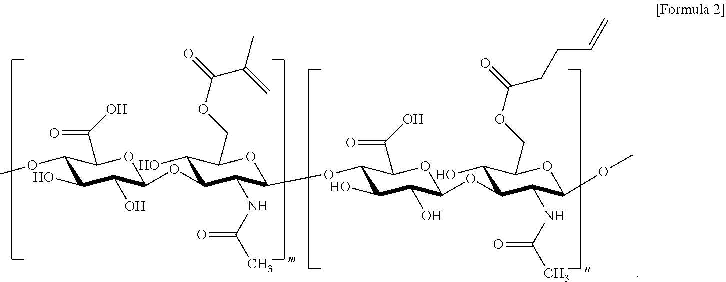

The step (S1A) is a step for preparing a first solution by dissolving hyaluronic acid (HA) in water (H₂O) and may further comprise cooling the first solution at 0 to 5° C.

The first solution may comprise hyaluronic acid and water in a weight ratio of 1:4 to 100 (hyaluronic acid:water), preferably, comprise hyaluronic acid and water in a weight ratio of 1:4 to 20 (hyaluronic acid:water), and most preferably, comprise hyaluronic acid and water in a weight ratio of 1 8 to 12 (hyaluronic acid:water).

The step (S1B) may be a step for preparing a second solution by adding a compound having a photo-cross-linking functional group and a base to the first solution prepared in the step (S1A).

The compound having a photo-cross-linking functional group may be at least one selected from the group consisting of methacrylic anhydride, acrylic anhydride, 3-butenoic anhydride, 4-pentenoic anhydride, 5-hexenoic anhydride, 6-heptenoic anhydride, 7-octenoic anhydride, 8-nonenoic anhydride, and 9-decenoic anhydride.

The compound having a photo-cross-linking functional group is bonded to the compound represented by [Formula 1] above to form a polymer through a photo-cross-linking reaction to form a structure of a photo-cross-linking copolymerized hyaluronic acid sponge.

The base may be at least one selected from the group consisting of sodium hydroxide (NaOH), sodium carbonate (Na₂CO₃), sodium bicarbonate (NaHCO₃), potassium hydroxide (KOH), potassium carbonate (K₂CO₃), and potassium bicarbonate (KHCO₃), preferably, sodium hydroxide or potassium hydroxide having a concentration of 1 to 5 M, and most preferably, sodium hydroxide or potassium hydroxide having a concentration of 1 to 3 M.

By adding a base in the step (S1B), the anhydride bond of the compound having a photo-cross-linking functional group is weakened to induce a polymerization reaction between a hydroxy (—OH) group bound to hyaluronic acid and the compound having a photo-cross-linking functional group so that a new ester bond may be generated.

In the step (S1B), the add may be added to maintain the pH at 5 to 9, preferably, at 6 to 8. The pH may be maintained neutral so that the reaction can be effectively performed to generate an ester bond of the compound represented by [Formula 1].

The step (S1C) may be a step for preparing a compound represented by [Formula 1] above by adding an alcohol to the second solution prepared in the step (S1B) to form a precipitate.

The alcohol may be a branched or straight chain C₁ to C₄ lower alcohol, preferably, at least one selected from the group consisting of methanol, ethanol, isopropanol, and 1,4-butanol, and most preferably, at least one selected from the group consisting of ethanol and isopropanol.

The step (S1C) may additionally comprise drying the precipitate. The drying may be vacuum drying, reduced pressure drying, boiling drying, hot air drying, spray drying or freeze-drying, preferably, vacuum drying or reduced pressure drying, and most preferably, vacuum drying.

The step (S2) is a step for preparing a photo-cross-linking copolymerized hyaluronic acid sponge according to the present invention and may consist of steps described below:

(S2A) preparing an aqueous solution by adding a photoinitiator and water (H₂O) to the compound represented by [Formula 1] above;

(S2B) irradiating ultraviolet (UV) ray to the aqueous solution to perform a photo-cross-linking reaction; and

(S2C) after the photo-cross-linking reaction, freeze-drying at −40 to 40° C. to prepare a photo-cross-linking copolymerized hyaluronic acid sponge.

More specifically, the step (S2A) may be a step for preparing an aqueous solution by adding a photoinitiator and water (H₂O) to the compound represented by [Formula 1] above prepared in the step (S1).

The photoinitiator may be at least one selected from the group consisting of 2-hydroxy-1-[4-(2-hydroxyethoxy)phenyl] 2-methyl-1-propanone, 3-(4-benzoylphenoxy)-2-hydroxy-N,N,N-trimethyl-1-propanaminium-chloride, methyl diethanolamine, sodium phenyl-2,4,6-trimethylbenzoylphosphinate, lithium phenyl-2,4,6-trimethylbenzoylphosphinate, benzyldimethyl ketal, and 1-hydroxycyclohexyl phenyl ketone.

The photoinitiator may react with the photo-cross-linking functional group bonded to the compound represented by [Formula 1] above to initiate a polymerization reaction by UV irradiation.

In the step (S2A), the compound represented by [Formula 1]:the photoinitiator may be mixed at a weight ratio of 1 to 100:1, preferably, the compound represented by [Formula 1]:the photoinitiator may be mixed at a weight ratio of 5 to 50:1, and most preferably, the compound represented by [Formula 1]:the photoinitiator may be mixed at a weight ratio of 10 to 30:1.

The step (S2B) may be a step for irradiating ultraviolet (UV) ray to the aqueous solution prepared in the step (S2A) to perform a photo-cross-linking reaction and the UV may be 300 to 500 nm, preferably, 350 to 450 nm.

The step (S2C) may be a step for, after performing the step (S2B), freeze-drying at −40 to 40° C. to prepare a photo-cross-linking copolymerized hyaluronic acid sponge.

The freeze-drying may be performed for 12 to 48 hours, preferably, 18 to 36 hours, but is not limited thereto.

The freeze-drying may be performed at a pressure of 1 to 100 mTorr, preferably, at a pressure of 1 to 50 mTorr.

### Use of Photo-Cross-Linked Hyaluronic Acid Sponge

The present invention can be used for medical antihemorrhagic agents, wound-coating agents, and adhesive barriers comprising the photo-cross-linking copolymerized hyaluronic acid sponge, and can be easily applied as long as it can be used as a biodegradable medical material without being limited thereto.

The photo-cross-linking copolymerized hyaluronic acid sponge comprising the compound represented by [Formula 1] above and the preparation method therefor are as defined above, and all the matters mentioned above are equally applied unless they contradict with each other.

Advantages and features of the present invention and methods for achieving the same will become clear with reference to the Examples described in details below with the accompanying drawings. However, the present invention is not limited to the Examples disclosed below, but will be implemented in a variety of different forms. These Examples make the disclosure of the present invention complete and are provided to completely inform the scope of the invention to one of ordinary skill in the art. The present invention is defined only by the scope of the claims.

Reagents and solvents mentioned below were purchased from Sigma Aldrich Korea unless otherwise specified, and ¹H NMR was measured by using a Varian Gemini-300 (300 MHz) spectrometer (Palo Alto, California, USA).

### Example 1. Photo-Cross-Linking Copolymerized Hyaluronic Acid Sponge

### 1.1 Preparation of Compound Represented by [Formula 2]

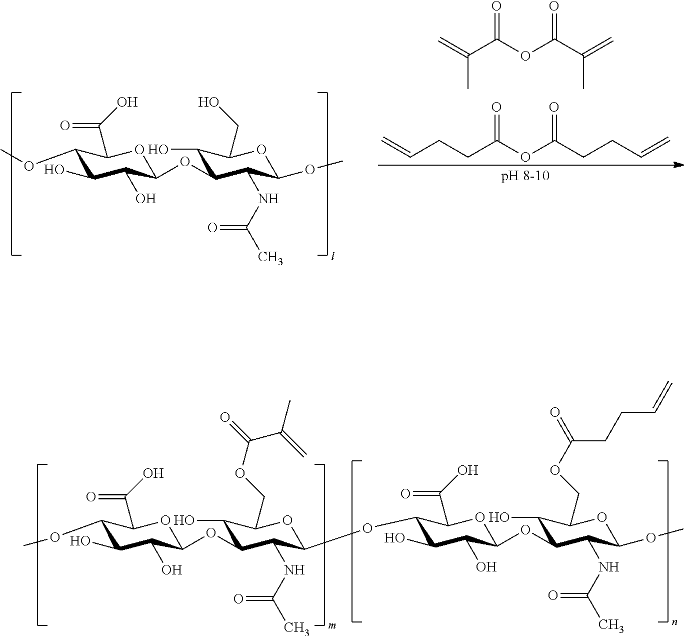

A first solution was prepared by adding 10 g of hyaluronic acid (HA) (26 mmol) to 100 ml of water (H₂O), and methacrylic anhydride, 3-butenoic anhydride, and 3 M NaOH were added to the first solution to prepare a second solution. In addition, ethanol was added to the second solution to produce a precipitate, and the precipitate was purified and vacuum dried to prepare a compound represented by [Formula 2].

¹H NMR (300 MHz, D₂O): δ(ppm)=5.9, 5.6, (2H, —C=CH₂), 5.7, 4.9, 4.8, (3H, —CH=CH₂), 4.5-4.3 (2H, CH₂), 3.8-3.0(10H, CH), 2.4-2.2 (2H, CH₂), 1.7 (3H, CH₃), 1.8 (3H, CH₃).

Yield: 83%.

### 1.2 Photo-Cross-Linking Copolymerized Hyaluronic Acid Sponge Comprising a Compound Represented by [Formula 2]

The compound represented by [Formula 2] prepared above and a photoinitiator (2-hydroxy-1-[4-(2-hydroxyethoxy)phenyl]2-methyl-1-propanone) were mixed at a weight ratio of 20:1, and the resulting mixture was added to water to prepare an aqueous solution. In addition, UV ray within a wavelength range of 365 nm was irradiated to the aqueous solution to perform a photo-cross-linking reaction, and freeze-drying was performed at −20° C. and 5 mTorr to prepare a photo-cross-linked hyaluronic acid sponge comprising a compound represented by [Formula 2].

### Comparative Example 1. Preparing a Comparative Photo-Cross-Linked Hyaluronic Acid Sponge

A compound represented by [Formula 3] below and a photoinitiator (2-hydroxy-1-[4-(2-hydroxyethoxy)phenyl]2-methyl-1-propanone) were mixed at a weight ratio of 20:1, and the resulting mixture was added to water to prepare an aqueous solution. In addition, UV ray within a wavelength range of 365 nm was irradiated to the aqueous solution to perform a photo-cross-linking reaction, and freeze-drying was performed at −20° C. and 5 mTorr to prepare a comparative photo-cross-linked hyaluronic acid sponge comprising a compound represented by [Formula 3].

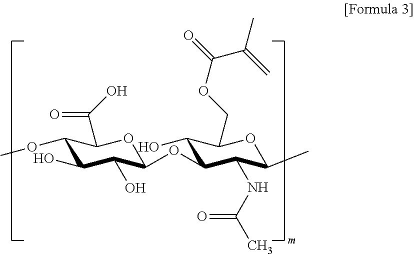

### Experimental Example 1. Confirming Water Binding Force

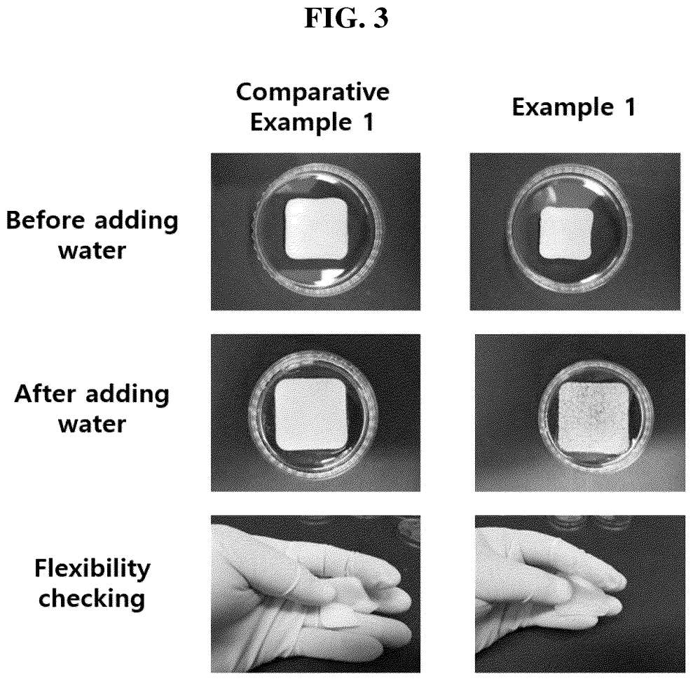

To confirm the hygroscopicity, water-binding ability, and flexibility of the hyaluronic acid, an experiment described below was performed. First, water was individually dropped on the photo-cross-linked hyaluronic acid sponge prepared in Example 1 and the comparative photo-cross-linked hyaluronic acid sponge prepared in Comparative Example 1, which were checked 30 minutes later. The results are shown in FIG. 3.

Referring to FIG. 3, in the case of the comparative photo-cross-linked hyaluronic acid sponge prepared in Comparative Example 1, the length of the alkyl group of the reactive group was short, which shortened the cross-linking length, and so the cross-linked sponge exhibited a hard property. Therefore, when it was brought into contact with moisture and bent, it was broken due to lack of flexibility. On the other hand, in the case of the photo-cross-linking copolymerized hyaluronic acid sponge prepared in Example 1 according to the present invention, a cross-linking agent having a longer alkyl group was added, which increased the cross-linking length, and so the cross-linked sponge exhibited more flexibility than that of Comparative Example 1.

The results described above show that when attached to a skin after a surgery or an wounded skin, the photo-cross-linking copolymerized hyaluronic acid sponge according to the present invention can absorb blood or exudate, can have a more flexible shape, and can have an advantage in providing a moist environment, thus helping organ or skin regeneration.

From the description above, one of ordinary skill the art pertaining to the present invention will be able to understand that the present invention can be implemented in other specific forms without changing the technical principles or essential features thereof. In this regard, the Examples described above are illustrative in all respects and should be understood as non-limiting.

1. A photo-cross-linking copolymerized hyaluronic acid sponge comprising a compound represented by [Formula 1] below: 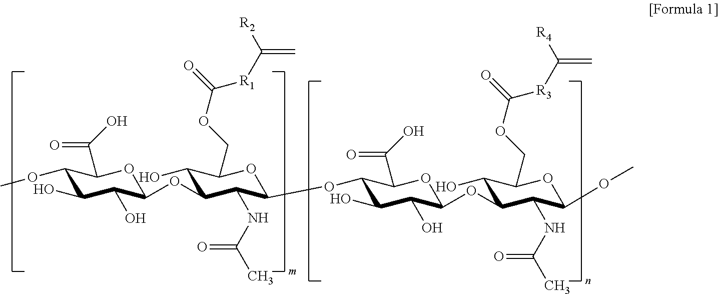
wherein, in [Formula 1] above,
the R1 and R3 are each independently a C₀-C₇ alkyl group,
the R2 and R4 are each independently a C₀-C₁ alkyl group, and
the m and n are each independently a positive integer of 1 to

2. The photo-cross-linking copolymerized hyaluronic acid sponge according to claim 1, wherein the compound represented by [Formula 1] is characterized in that it is a compound represented by [Formula 2] below:

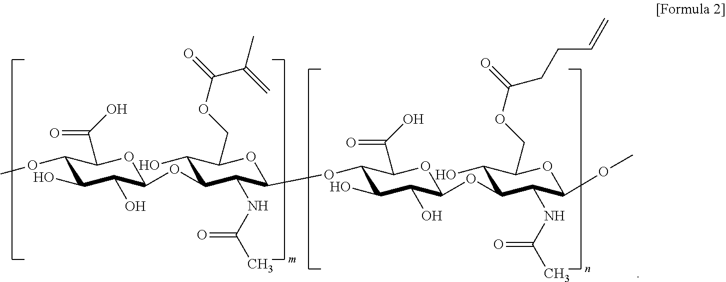

3. A preparation method for a photo-cross-linked hyaluronic acid sponge comprising:
(S1) preparing a compound represented by [Formula 1] below; and
(S2) preparing a photo-cross-linked hyaluronic acid sponge by adding a photoinitiator to the compound represented by the [Formula 1], 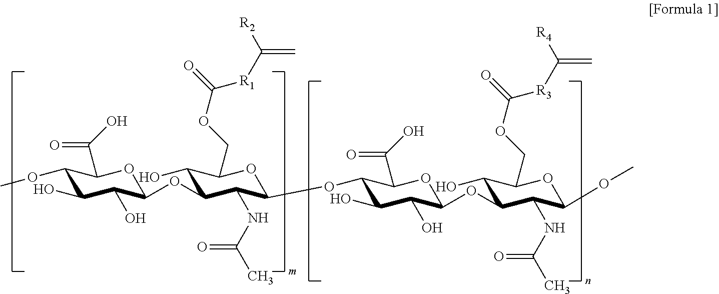
wherein, in [Formula 1] above,
the R1 and R3 are each independently a C₀-C₇ alkyl group,
the R2 and R4 are each independently a C₀-C₁ alkyl group, and
the m and n are each independently a positive integer of 1 to 10,000.

4. The preparation method for a photo-cross-linked hyaluronic acid sponge according to claim 1, wherein the step (S1) is characterized in that it consists of:
(S1A) preparing a first solution by dissolving hyaluronic acid (HA) in water (H₂O);
(S1B) preparing a second solution by adding a compound having a photo-cross-linking functional group and a base to the first solution; and
(S1C) preparing a compound represented by [Formula 1] by adding an alcohol to the second solution to form a precipitate.

5. The preparation method for a photo-cross-linked hyaluronic acid sponge according to claim 4, wherein the compound having a photo-cross-linking functional group is at least one selected from the group consisting of methacrylic anhydride, acrylic anhydride, 3-butenoic anhydride, 4-pentenoic anhydride, 5-hexenoic anhydride, 6-heptenoic anhydride, 7-octenoic anhydride, 8-nonenoic anhydride, and 9-decenoic anhydride.

6. The preparation method for a photo-cross-linked hyaluronic acid sponge according to claim 3, wherein the compound represented by [Formula 1] above is characterized in that it is a compound represented by [Formula 2] below:

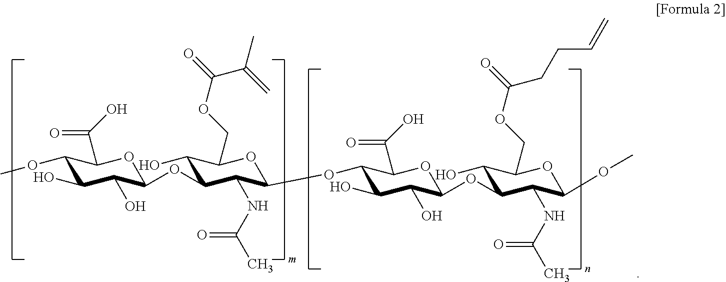

7. The preparation method for a photo-cross-linked hyaluronic acid sponge according to claim 3, wherein the step (S2) is characterized in that it consists of steps described below:
(S2A) preparing an aqueous solution by adding a photoinitiator and water (H₂O) to the compound represented by [Formula 1] above;
(S2B) irradiating ultraviolet (UV) ray to the aqueous solution to perform a photo-cross-linking reaction; and
(S2C) after the photo-cross-linking reaction, freeze-drying at −40 to 40° C. to prepare a photo-cross-linked hyaluronic acid sponge.

8. The preparation method for a photo-cross-linked hyaluronic acid sponge according to claim 7, wherein the photoinitiator is characterized in that it is at least one selected from the group consisting of 2-hydroxy-1-[4-(2-hydroxyethoxy) phenyl] 2-methyl-1-propanone, 3-(4-benzoylphenoxy)-2-hydroxy-N,N,N-trimethyl-1-propanaminium-chloride, methyl diethanolamine, sodium phenyl-2,4,6-trimethylbenzoylphosphinate, lithium phenyl-2,4,6-trimethylbenzoylphosphinate, benzyldimethyl ketal, and 1-hydroxycyclohexyl phenyl ketone.
